(19) United States
(12) Patent Application Publication
(10) Pub. No.: US 2025/0312004 A1
(43) Pub. Date: Oct. 9, 2025

(54) NEAR FIELD ULTRASOUND MEASURING SYSTEMS AND METHODS

(71) Applicant: Provisio Medical, Inc., San Diego, CA (US)

(72) Inventors: Stephen Eric Ryan, San Diego, CA (US); Nestor E. Cabrera-Munoz, San Diego, CA (US)

(21) Appl. No.: 18/873,111

(22) PCT Filed: Jun. 16, 2023
(86) PCT No.: PCT/US2023/025615

§ 371 Date: Dec. 9, 2024

Related U.S. Application Data
(60) Provisional application No. 63/355,507, filed on Jun. 24, 2022.

(51) Int. Cl. A61B 8/00; A61B 8/08; A61B 8/12

(52) U.S. Cl. CPC A61B 8/4254; A61B 8/085; A61B 8/0891; A61B 8/12; A61B 8/4477; A61B 8/4494

(57) ABSTRACT

A method of ultrasound measuring including transmitting ultrasound signals from a plurality of ultrasound transducers toward a structure at a first resonant frequency of the transducers. Ultrasound signals are received from the structure in response to the transmitted ultrasound signals and a determination is made of whether each of the plurality of transducers is within a proximity threshold of the structure. Based on determining that a transducer is within the threshold, ultrasound signals are transmitted from the transducer toward the structure at a second resonant frequency lower than the first resonant frequency. A distance between each of the plurality of transducers and the structure is calculated based on the ultrasound signals received from the structure in response to the transmitted ultrasound signals at the first resonant frequency and/or second resonant frequency and the shape and metrics of the structure are determined.

### REFERENCE TO RELATED APPLICATIONS

This application claims the benefit of U.S. Provisional Application No. 63/355,507, filed Jun. 24, 2022, which is hereby incorporated by reference in its entirety.

### BACKGROUND

### Field of the Disclosure

The present disclosure relates generally to systems, methods, and devices that utilize ultrasound to gather dimensional and physiological information about structures such as fluid-filled body vessels.

### Description of Related Art

Recent studies have illustrated that the predominate cause of endovascular treatment failure is inaccurate sizing of vessels or inadequate treatment to achieve the lumen dimensions desired over an entire stenotic lesion. An improperly selected, dimensioned, and/or positioned medical device (e.g., a stent) and/or treatment can lead to highly adverse outcomes including avoidable death. Typical techniques used for analyzing the structural features of blood vessels include angiography. However, angiography only provides limited and imprecise information about the size and morphology of blood vessels and often does not allow the physician to adequately assess the lesion prior to treatment. Thus there is a need for systems, methods, and devices to gather dimensional and physiological information about structures such as fluid-filled body vessels.

### SUMMARY

Embodiments of the present disclosure include a novel implementation of an ultrasound measurement probe to approximate the dimensions and/or shape(s) of fluid-filled structures. Some embodiments include an elongated flexible body such as a catheter with multiple ultrasound transducers arranged circumferentially about the catheter for generating and receiving ultrasound signals to and from the surrounding structure. The transducers are configured for and activated at an optimal design center frequency for obtaining ultrasound distance measurements between the probe and walls of the structure (e.g., using time-of-flight such as further referenced herein). Generally, higher design center frequencies provide more accuracy with better temporal-spatial resolutions but less depth of penetration, since ultrasound signal attenuation in surrounding media (e.g., blood) and structures (e.g., blood vessel wall) proportionally increases with frequency. Moreover, higher frequency transducers are also more susceptible to imaging artifacts produced by higher intensity side lobes that can result in noisy signals.

In order to maximize distance measurement accuracy without the need to penetrate tissue, the transducers are configured with a frequency of at least about 10 MHz such as, for example, about 20 MHz or 30 MHz. As the elongated flexible body is moved within or about a structure, the transducers collect data at different positions with respect to the structure. As one or more of the transducers are positioned in close proximity to the structure, the excitation pulse and responsive return signals at those transducers become closer in time and more difficult to differentiate, particularly if the transducers are positioned directly against the structure.

In some embodiments, a method for processing ultrasound signals when a probe's transducers are within a proximity threshold of a structure includes transmitting ultrasound signals toward the structure using the transducers' fundamental resonant frequency (i.e., first harmonic frequency) and determining whether the structure is within the threshold. When one or more of the transducers are determined to be within the threshold, they are switched to operating at a lower resonant frequency (e.g., a half harmonic frequency of the transducers) and used to obtain additional measurements. For example, a 30 MHz transducer may be transitioned to resonate at a half harmonic of 15 MHz. For a given transducer aperture size, the lower frequency reduces the noise level of the transducer(s) by reducing the intensity of the ultrasound beam sidelobes; the main lobe becomes wider, making it less susceptible to motion related artifacts from particles (e.g., blood cells) travelling in the surrounding medium (e.g., blood); and the focal point is brought closer, extending the Fraunhofer zone (i.e., far field) where the ultrasound beam radiation pattern is well defined and useful. Furthermore, by lowering the frequency, there are less excitation cycles activating the transducer(s) for a given duration of time, bringing the excitation signal intensity down, and subsequently shortening the excitation signal total pulse width at a determined intensity level, as discussed further herein. As a result, the captured return signals are less likely to be interfered by the excitation signal and can be used to measure the distance(s) between the transducer(s) and the structure more effectively.

After distance measurements for each of the transducers are obtained, the measurements may be used to determine a cross-section of the structure by fitting curves to points of the structure wall that are based on the distance measurements.

In some embodiments, determining whether a transducer is within a proximity threshold includes monitoring the relative position between a transducer excitation signal pulse and a structure wall peak in the return signals. In some embodiments, when a structure wall peak cannot be distinguished from other signals, the transducer is determined to be within the proximity threshold and transitioned to the lower frequency for further measurements. In some embodiments, the interval between the pulse and structure wall peak is monitored and compared over successive measurements. A determination that the transducer is within the proximity threshold may be made when the successive measurements show that transducer approached the threshold prior to the signal “merging” with the excitation pulse.

In some embodiments, where measurements from some transducers can be obtained at a particular position of the probe and where measurements from other transducers cannot be sufficiently obtained, a preliminary cross-section of the structure is calculated based on the obtained measurements and this cross-section is used to determine whether transducers are within the proximity threshold. Those transducers determined to be within the proximity threshold are transitioned to operating at the lower frequency. After transitioning to the lower frequency, measurements from the transitioned transducers are used to complete the determination of a complete cross-section calculation of the structure.

In some embodiments, a method for ultrasound measuring includes transmitting ultrasound signals toward a structure from each of a plurality of ultrasound transducers centered at a first resonant frequency and centered at a second resonant frequency, the second resonant frequency lower than the first resonant frequency; receiving ultrasound signals from the structure responsive to the transmitted ultrasound signals; determining distances between each of the plurality of transducers and the structure based on the received ultrasound signals; for each determined distance, calculating a circumferentially separated coordinate point of the structure based on the respective determined distance between the respective transducer and structure; and generating a computed map image in a computer display that includes calculating and plotting a cross-sectional map of the structure by interpolating between the circumferentially separated coordinate points of the structure each based on the respective determined distance.

In some embodiments, the first resonant frequency is the fundamental/first harmonic of the transducers. In some embodiments, the second resonant frequency is a half harmonic of the transducers. In some embodiments, the first resonant frequency is about 30 MHz and the second resonant frequency is about 15 MHz.

In some embodiments, the method for ultrasound measuring further includes determining that one or more of the plurality of transducers is within a proximity threshold of the structure; in response to determining that the one or more of the plurality of transducers are within the proximity threshold, determining the distances between the one or more transducers and the structure based on the ultrasound signals responsive to the second resonant frequency. In some embodiments, determining that one or more of the plurality of transducers is within a proximity threshold includes identifying an absence of a structure signal peak separated by more than a predetermined time interval from an excitation pulse signal. In some embodiments, identifying an absence of a separated structure signal peak includes determining that a structure signal peak has substantially merged with the excitation pulse signal. In some embodiments, determining that one or more of the plurality of transducers is within the proximity threshold includes, based on the received ultrasound signals, calculating a distance between each of a first subset of the plurality of transducers and the structure; for each calculated distance, calculating a circumferentially separated coordinate point of the structure based on the respective determined distance between the respective transducer and the structure; determining a partially-calculated cross-section of the structure based on the separated coordinate points for the first subset of transducers; estimating a distance between each of a second subset of the plurality of transducers and the structure based on the partially-calculated cross-section, the second subset including one or more transducers not within the first subset; and determining that the second subset of transducers is within the proximity threshold based on the estimation of their distance from the structure. In some embodiments, estimating the distance between each of the second subset of transducers and the structure includes calculating the length of a radial distance line between a position of each of the subset of transducers and the structure based on the partially-calculated cross-section. In some embodiments, the proximity threshold is about 0.3 millimeters or less. In some embodiments, the proximity threshold is about 0.2 millimeters or less. In some embodiments, the proximity threshold is about 0.1 millimeters or less. In some embodiments, the method of ultrasound measuring further includes determining that one or more of the plurality of transducers is not within the proximity threshold of the structure; and in response to determining that one or more of the plurality of transducers is not within the proximity threshold of the structure, calculating the distances between the transducers not within the proximity threshold and the structure based on the first resonant frequency.

In some embodiments, calculating a distance between each of the plurality of transducers and the structure is based on the ultrasound signals received from the structure in response to a combination of ultrasound signals transmitted at the first resonant frequency and the second resonant frequency. In some embodiments, a distance between a transducer and the structure is calculated to be about zero based on determining, from the received signals, that the transducer is within a proximity threshold of the structure. In some embodiments, determining that the transducer is within a proximity threshold includes determining that a wall signal peak within the received signals has substantially merged with an excitation pulse of the received signals. In some embodiments, calculating a distance between a transducer and the structure is based on determining the stability of the signals using each of the first and second resonant frequencies received from the structure and selecting the signals determined to be more stable to calculate the respective distance. In some embodiments, calculating a distance between a transducer and the structure is based on signals of at least one of the first or second resonant frequencies and by verifying the distance calculation using signals of the other of the at least one of the first or second resonant frequencies. In some embodiments, calculating a distance between each of the transducers and the structure is based on: in response to determining that the respective transducer is within a proximity threshold of the structure, calculating the distance based on signals using the second resonant frequency; and in response to determining that the respective transducer is not within a proximity threshold of the structure, calculating the distance based on signals using the first resonant frequency.

In some embodiments, the duration of ultrasound signal transmission at the second resonant frequency is equal to the duration of ultrasound signal transmission at the first resonant frequency. In some embodiments, the duration of ultrasound signal transmission at the second resonant frequency is no greater than about the duration of ultrasound signal transmission at the first resonant frequency. In some embodiments, the duration of ultrasound signal transmission at the second resonant frequency is shorter than the duration of ultrasound signal transmission at the first resonant frequency. In some embodiments, the one or more transducers generate lower noise levels in response to transmitting at the second resonant frequency compared to noise levels generated in response to transmitting at the first resonant frequency. In some embodiments, transmitting the ultrasound signals includes generating a main ultrasound beam and side lobes of the main beam, wherein the main beam is wider and the side lobes less intense using the second resonant frequency compared to using the first resonant frequency. In some embodiments, transmitting the ultrasound signals includes generating a wider excitation pulse width at the first resonant frequency compared to the second resonant frequency at and above a particular intensity level.

In some embodiments, the particular intensity level is at least above a noise floor. In some embodiments, the particular intensity level is about −20 dB or greater. In some embodiments, the particular intensity level is about −60 dB or greater. In some embodiments, the particular intensity level is about −100 dB or greater. In some embodiments, generating a narrower excitation pulse at and above a particular intensity level includes transmitting the ultrasound signals at a lower intensity for the second resonant frequency compared to the first resonant frequency. In some embodiments, generating a narrower excitation pulse at and above a particular intensity level comprises transmitting the ultrasound signals with a lower number of excitation signal pulses for the second resonant frequency compared to the first resonant frequency.

In some embodiments, each of the plurality of ultrasound transducers is circumferentially separated from each other; transmitting ultrasound signals toward a structure from each of the plurality of ultrasound transducers includes transmitting substantially orthogonally a signal from each transducer toward a respectively separated circumferential portion of the structure substantially parallel to the transducer at the first resonant frequency and at the second resonant frequency; and receiving ultrasound signals from the structure responsive to the transmitted ultrasound signals comprises receiving at each separated transducer a reflected signal from the respectively circumferentially separated section of the structure.

In some embodiments, an ultrasound system for measuring the dimensions of a structure includes a flexible body elongated along a longitudinal axis and assembled for insertion into the structure; a plurality of ultrasound transducers arranged on the flexible body; and one or more processors programmed and configured to cause: transmitting ultrasound signals from a plurality of ultrasound transducers of an ultrasound probe toward a structure at a first resonant frequency; and receiving responsive ultrasound signals at the ultrasound transducers responsive to the respective sets of transmitted ultrasound signals; and in response to determining that one or more of the plurality of transducers is within the proximity threshold: transmitting ultrasound signals from the one or more transducers toward the structure at a second resonant frequency lower than the first resonant frequency; receiving ultrasound signals from the structure responsive to the transmitted ultrasound signals at the second resonant frequency; and calculating a distance between each of the plurality of transducers and the structure based on the ultrasound signals received from the structure in response to the transmitted ultrasound signals at the first resonant frequency and/or second resonant frequency.

In some embodiments, an ultrasound system for measuring the dimensions of a structure includes a flexible body elongated along a longitudinal axis and assembled for insertion into the structure; a plurality of ultrasound transducers arranged on the flexible body; and one or more processors programmed and configured to cause: transmitting ultrasound signals toward a structure from each of a plurality of ultrasound transducers centered at a first resonant frequency and centered at a second resonant frequency, the second resonant frequency lower than the first resonant frequency; receiving ultrasound signals from the structure responsive to the transmitted ultrasound signals; determining distances between each of the plurality of transducers and the structure based on the received ultrasound signals; for each determined distance, calculating a circumferentially separated coordinate point of the structure based on the respective determined distance between the respective transducer and structure; generating a computed map image in a computer display that comprises calculating and plotting a cross-sectional map of the structure by interpolating between the circumferentially separated coordinate points of the structure each based on the respective determined distance; and generating a computed map image in a computer display that comprises combining and plotting a series of multiple previously calculated cross-sectional maps at multiple longitudinal and lateral positions within the structure to generate a three-dimensional mapping representation of the structure.

In some embodiments, a method of ultrasound measuring includes transmitting ultrasound signals from a plurality of ultrasound transducers toward a structure using least one resonant frequency of the transducers, the at least one resonant frequency including a first resonant frequency; receiving ultrasound signals from the structure responsive to the transmitted ultrasound signals; based on the received ultrasound signals, determining whether the structure is within a proximity threshold of each of the plurality of transducers; calculating distances between each of the plurality of transducers and the structure based on the received ultrasound signals; wherein, in response to determining that one or more of the plurality of transducers is within the proximity threshold, the calculating of distances between the structure and each transducer within the proximity threshold is based on ultrasound signals transmitted at a second resonant frequency lower than a first resonant frequency of the at least one resonant frequency; transmitting ultrasound signals from the one or more transducers toward the structure at a second resonant frequency lower than the first resonant frequency; receiving ultrasound signals from the structure responsive to the transmitted ultrasound signals at the second resonant frequency; and calculating a distance between each of the plurality of transducers and the structure based on the ultrasound signals received from the structure in response to the transmitted ultrasound signals at the first resonant frequency and/or second resonant frequency.

### BRIEF DESCRIPTION OF THE DRAWINGS

Embodiments of the disclosure will be described hereafter in detail with particular reference to the drawings. Throughout this description, like elements, in whatever embodiment described, refer to common elements wherever referred to and referenced by the same reference number. The characteristics, attributes, functions and interrelations ascribed to a particular element in one location apply to that element when referred to by the same reference number in another location unless specifically stated otherwise. In addition, the exact dimensions and dimensional proportions to conform to specific force, weight, strength and similar requirements will be within the skill of the art after the following description has been read and understood.

All figures are drawn for ease of explanation of the basic teachings of the present disclosure only; the extensions of the figures with respect to number, position, relationship and dimensions of the parts to form examples of the various embodiments will be explained or will be within the skill of the art after the present disclosure has been read and understood.

FIG. 1 is an illustrative diagram of an ultrasound catheter probe system according to some embodiments.

FIG. 2A is an illustrative side perspective diagram of an ultrasound catheter probe placed within a lumen at different positions according to some embodiments.

FIG. 2B are cross-sectional perspective diagrams of the ultrasound catheter probe of FIG. 2A according to some embodiments.

FIG. 2C is another cross-sectional perspective diagram of the ultrasound catheter probe across lines I-I′ of FIG. 2A but only depicts one of either 15A or 15B.

FIG. 3A is an illustration of ultrasound transducer signal intensities over time at different operating frequencies according to some embodiments.

FIG. 3B is an illustration of ultrasound transducer signal intensities over time at different operating frequencies when the transducer is positioned closer to a structure relative to that shown in FIG. 3A according to some embodiments.

FIG. 3C is an illustration of ultrasound transducer signal intensities over time at different operating frequencies when the transducer is positioned substantially adjacent to the structure shown in FIGS. 3A and 3B according to some embodiments.

FIG. 4A is an illustrative cross-sectional diagram of an ultrasound transducer probe array positioned within a lumen structure while operating at fundamental frequency according to some embodiments.

FIG. 4B is an illustrative partially-calculated cross-sectional map of the lumen structure generated from distance measurements obtained through the ultrasound transducer probe of FIG. 4A according to some embodiments.

FIG. 4C is an illustrative cross-sectional diagram of an ultrasound transducer probe array positioned within a lumen structure while operating at a lower half harmonic frequency relative to FIG. 4A according to some embodiments.

FIG. 4D is an illustrative completely-calculated cross-sectional map of the lumen structure generated from distance measurements obtained through the ultrasound transducer probe of FIG. 4C according to some embodiments.

FIG. 5 is a flow chart of a process for utilizing multiple resonant frequencies for calculating structure dimensions according to some embodiments.

FIG. 6 is a flow chart of a process for monitoring the proximity of transducers to a structure in order to transition them between high and lower resonant frequencies according to some embodiments.

FIG. 7A is an illustrative cross-sectional view of an ultrasound probe moved within various positions of a lumen according to some embodiments.

FIG. 7B is an illustrative three-dimensional perspective image of the lumen of FIG. 7A generated from an ultrasound probe according to some embodiments.

### DETAILED DESCRIPTION

Obtaining and utilizing structural information about patients is a critical aspect of diagnosing and treating many medical conditions. For example, within the field of endovascular medicine, it is important to gain structural and physiological information about diseased blood vessels when selecting among interventional techniques such as angioplasty, stents, and/or surgery. Recent studies have shown that outcomes are significantly improved through the use of more advanced, more accurate imaging techniques.

Some imaging catheters utilize ultrasound or optical technologies to provide a more accurate cross-sectional imaging that may then be interpreted by the physician to determine, among other characteristics, the dimensions of the lumen surrounding the catheter. For example, Intravascular Ultrasound (IVUS) and Optical Coherence Tomography (OCT) have been used in interventional diagnostic procedures to image blood vessels to locate and characterize atherosclerosis and other vessel diseases and defects.

IVUS and OCT images can be used to determine information about a vessel, including vessel dimensions, and is typically much more detailed than the information that is obtainable from traditional angiography images, which are generally limited to two-dimensional shadow images of the vessel lumen. The information gained from more accurate imaging techniques can be used to better assess physiological conditions, select particular procedures, and/or improve performance of the procedure.

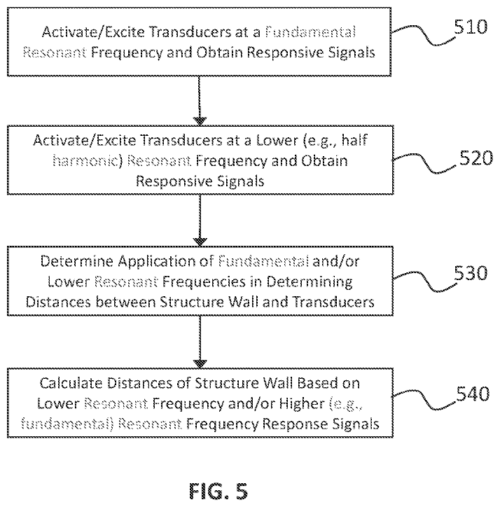

While current IVUS and OCT systems provide additional and more detailed information compared to angiograms, these IVUS and OCT systems are limited by the size of the structures (e.g., by the diameters of target blood vessels) in which they can be placed and perform imaging without being substantially and detrimentally impacted by the size/proximity of the structure. For example, the focal length of the imagers, signal noise, and other interference will impact traditional OCT/IVUS imagers when the target structures fall within close range of the imagers. That is why many such imaging catheter probes require a feature for centering the probes positioned within a lumen structure (e.g., using a vessel-centering balloon or other feature as illustrated in FIGS. 5-10 of U.S. Patent Application Publication No. US 20160051323 A1 by Stigall, et al.)

The components of these systems (e.g., transducers, wires, imaging circuitry, fiber-optics, probe-centering features, etc.) can occupy a large footprint within the blood vessel and further increase the minimum size of vessels in which these imaging probes can be placed. Further, the images produced by IVUS and OCT systems may not directly provide useful information about blood vessels and are typically subject to nonconforming interpretations of different physicians. Thus, there is a need for an improved and more efficient way to get reliable needed information about a vessel or structure (e.g., diameters, area, volume, and multi-dimensional profile), including small-sized vessels, without the need for additional vessel-centering components, while not sacrificing speed and footprint needed for timely, efficient, and effective treatment.

In order that embodiments of the disclosure may be clearly understood and readily carried into effect, certain embodiments of the disclosure will now be described in further detail with reference to the accompanying drawings. The description of these embodiments is given by way of example only and not to limit the scope of the disclosure.

FIG. 1 is an illustrative diagram of an ultrasound catheter probe system 28 according to some embodiments. In certain embodiments, an ultrasound imaging probe 10 includes a body 40 having a proximal end 14 and a distal end 16. In certain embodiments, the body 40 is elongated along a longitudinal axis. In certain embodiments, the probe 10 includes a plurality of transducers 18. In certain embodiments, the body 40 comprises an elongated tip 20 having a proximal end 22 and a distal end 24. In certain embodiments, the plurality of transducers 18 may be circumferentially distributed and separated about the probe 10. In some embodiments, the plurality transducers 18 are evenly distributed circumferentially on a holding body 50. In certain embodiments, the probe 10 includes a proximal connector 26 which connects the probe 10 to other components of the system 28, including a computer system 36. In certain embodiments, the medical device or probe 10 is part of a system 28 that includes a distal connector 30, electrical conductors 32, a data acquisition unit 34, and a computer system 36.

In some embodiments, the body 40 is tubular and includes a central lumen 38. In some embodiments, the body 40 has a diameter of about 1,500 μm, 650 μm, or less. These dimensions are illustrative and not intended to be limiting. In some embodiments, the diameter of the probe 10 will depend on the type of device that the probe 10 is integrated with and where the probe 10 will be used (e.g., in a blood vessel), which will become apparent to those of ordinary skill in the art in view of the present disclosure.

In certain embodiments, the proximal end 14 of the body 40 is attached to the proximal connector 26. In some embodiments, the probe 10 and the body 40 have an elongated tip 20 in which the proximal end 22 is attached to the distal end 16 of body 40. The elongated tip 20 may be constructed with an appropriate size, strength, and flexibility to be used for guiding the probe 10 through a body lumen (e.g., a blood vessel). The elongated tip 20 and/or other components of probe 10 may include one or more radiopaque markers (e.g., visible to angiography) for precisely guiding the catheter through a lumen and positioning the transducers 18 in the desired location. In some embodiments, probe 10 and distal end 16 are constructed and arranged for rapid exchange use. The body 40 and elongated tip 20 may be made of resilient flexible biocompatible material such as is common for IVUS and intravascular catheters known to those of ordinary skill in the art.

In some embodiments, the probe 10 may be integrated with an expandable balloon 43 (e.g., an angioplasty balloon). In some embodiments, the probe 10 and the body 40 may have multiple lumens for use with various features not shown (guidewires, fiberoptics, saline flush lumens, electrical connectors, etc.). In some embodiments, the outer diameter of the body 40 and the elongated tip 20, if present, is substantially consistent along its length and does not exceed a predetermined amount.

In some embodiments, the ultrasound transducers 18 are piezoelectric. In certain embodiments, the transducers are built using piezoelectric ceramic or crystal material, or composites of piezoelectric ceramic or crystal with polymers, and layered by one or more matching layers that can be thin layers of epoxy, epoxy composites/mixtures, or polymers. In some embodiments, the transducers are PMUTs (Piezoelectric Micromachined Ultrasonic Transducers), CMUTs (Capacitive Micromachined Ultrasonic Transducers), and/or photoacoustic transducers.

The operating frequency for the ultrasound transducers may be in the range of from about 8 MHz to about 50 MHz or even up to about 60 MHz, depending on the dimensions and characteristics of the transducer and requirements of the particular application. Generally, higher frequency of operation provides better resolution and a smaller medical device 10. However, the tradeoff for this higher resolution and smaller catheter size may be a reduced depth of penetration into the tissue of interest and increased echoes from the blood itself (making the image and/or measurements more difficult to interpret). Lower frequency of operation is more suitable for imaging in larger vessels or within structures such as the chambers of the heart. Although specific frequency ranges have been given, these ranges given are illustrative and not limiting. The ultrasonic transducers 18 may transmit and receive signals of any frequency that leaves one or more of the transducers 18, impinges on some structure or material of interest, and is reflected back to and picked up by one or more transducers 18. In some embodiments, the transmitted signals are directed toward circumferentially separated portions of the structure or material that is substantially parallel to the respective transducer.

The fundamental resonant frequency (center frequency) and bandwidth of a transducer is generally related to the thickness of transducer materials generating or responding to ultrasound signals. For example, in some embodiments, a transducer includes a piezoelectric material such as quartz and/or lead-zirconate-titanate (PZT). A thicker layer will generally respond to a longer wavelength and lower frequency and vice versa. For example, a 50-micron thick layer of PZT will have a fundamental resonant frequency of about 40 MHz, a 65-micron thick layer will have a fundamental resonant frequency of about 30 MHz, and a 100-micron thick layer will have a fundamental resonant frequency of about 20 MHz. As further described herein, matching and backing layers may be included in the one or more transducers 18 which affect the bandwidth and other characteristics of a transducer.

In some embodiments, probe 10 is connected with an actuating mechanism that may rotate and/or longitudinally move at least some portions of probe 10 and its transducers 18. A controlled longitudinal and/or radial movement permits the probe 10 to obtain ultrasound readings from different perspectives within a surrounding structure, for example. Positioning the probe 10 and its transducers in target locations may be augmented/guided by real-time imaging feedback provided by the transducers and system 28. Relative positions of the probe 10 may be tracked and recorded during such processes (e.g., by using an encoder or other position sensing tool).

In some embodiments, system 28 is programmed to analyze and identify characteristics of the medium (e.g., blood) between probe 10 and the structure in order to determine where the medium ends with respect to the structure (e.g., blood vessel wall). In some embodiments, multiple ultrasound measurements of the blood may be generated and the differences between the measurements are used to identify movement/change of the blood over time (e.g., as a result of a heart pumping). In some embodiments, Doppler echo signals are used to determine these differences. Because the blood vessel wall does not have the same movement/change characteristics as the blood, the amount (or distance) between the probe 10 and blood vessel wall can be calculated. In some cases, reliance on the blood measurements without substantial reliance on measurements of the blood vessel wall may be used to determine the distance between probe 10 and the blood vessel wall.

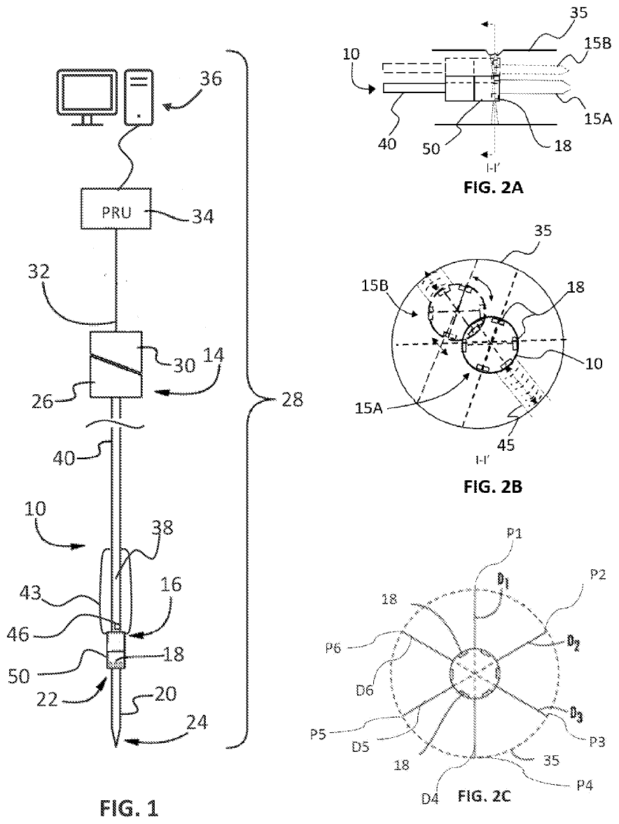

In certain embodiments, the computer system 36 is programmed to analyze and distinguish between the echoes associated with respective ultrasound pulses. In certain embodiments, the computer system 36 is programmed to analyze the signals and calculate a radial distance measurement between each transducer 18 and lumen 35. This may be done, for example, by utilizing time-of-flight information of the echo signals and previously determined/differentiated signatures representative of a lumen wall (e.g., of lumen 35 of FIG. 2A-2C) and a particular medium (e.g., blood) between the transducer 18 and lumen 35. Exemplary systems and methods for making such calculations are described, for example, in U.S. Pat. No. 10,231,701 filed Mar. 14, 2014 (the '701 Patent), the entire contents of which are herein incorporated by reference.

As described in the '701 Patent and below, based on the radial distance calculations (e.g., D1, D2, . . . , D6 of FIG. 2C and in FIG. 2 of the '701 Patent), the shape and dimensions of lumen 35 may be estimated by further utilizing information including the dimensions of the probe 10 and applying interpolation and/or other mathematical fitting techniques. For example, the relative positions of points (e.g., P1, . . . , P6, of FIG. 2C) about lumen 35 may first be calculated and a curve fitting algorithm (e.g., spline interpolation) is applied to generate a two-dimensional slice representation of the lumen 35. As described in the '701 Patent, multiple slices can be calculated by taking sets of ultrasound readings along the longitudinal extent of lumen 35 and combined to generate a three-dimensional representation. In some embodiments, one or more transducers 46 are positioned within balloon 43 and are used to calculate the level of expansion of balloon 43 as it is expanded, for example.

FIG. 2A is an illustrative side perspective diagram of an ultrasound catheter probe 10 placed within a lumen 35 at different positions according to some embodiments. FIG. 2B is a cross-sectional perspective diagram of the ultrasound catheter probe across lines I-I′ of FIG. 2A. Catheter probe 10 is shown inserted into a lumen 35 at positions 15A and 15B. Shifting positions can result from movement of probe 10 (e.g., mechanical actuation) and/or movement of lumen walls between positions 15A and 15B (e.g., from heart pumping, blood flow). FIG. 2C is another cross-sectional perspective diagram of the ultrasound catheter probe across lines I-I′ of FIG. 2A but only depicts one of either 15A or 15B.

The connected computer system 36 is programmed to cause the one or more transducers 18 to generate pulses (i.e., pulsed pressure waves) 45 where each of the pulses is incident on different circumferential portions of lumen 35 substantially along a radial line perpendicular to each transducer 18. In response to reflected pulses from lumen walls 35 at positions 15A and 15B, the transducers 18 generate electrical signals representing the pulses that reflect (i.e., echo) back from media and circumferential portions of lumen 35 adjacent and substantially parallel to each transducer 18 of probe 10. These electrical signals are then processed by a signal processor and computer system 36. In some embodiments, an envelope signal associated with the generated pulses 45 (i.e. excitation pulse) is detected and distinguished within the return signals to identify a transition between media and/or structural features. Based on the distinction, a distance measurement may be calculated (i.e. D1-D6) between the transducer/probe (18, 10) and the transition location along a line substantially perpendicular to probe 10. As discussed further herein, when a transducer of a probe is sufficiently proximate to a structure boundary (e.g., such as probe 10 at position 3C), signals associated with the excitation pulse and return signals may become substantially indistinguishable at a fundamental (i.e., first harmonic) operating frequency. In some embodiments, excitation pulses may be delivered simultaneously or at different times to transducers 18.

The computer system 36 is programmed to process these signals and calculate a radial distance measurement (D1-D6 of FIG. 2C) between each transducer 18 and lumen 35. In certain embodiments, this may be done, for example, by utilizing time-of-flight information of the echo signals and previously determined/differentiated signatures representative of a lumen wall (e.g., of a lumen wall 35 represented at different times and positions 15A and 15B) and a particular medium (e.g., blood) between the transducer 18 and lumen walls 35.

Based on distance calculations, the shape and dimensions of the lumen 35 may be estimated by further utilizing information including the dimensions of probe 10 and applying interpolation and/or other mathematical fitting techniques. For example, when sufficiently distinguishable return signals are obtained, the relative positions of points (p1-p6 of FIG. 2C) about the lumen wall 35 may first be calculated and a curve fitting algorithm (e.g., spline interpolation) is applied to generate a two-dimensional slice representation of the lumen based on signals from the wall. In certain embodiments, when one or more transducer(s) 18 become too close to a structure boundary (e.g., lumen wall 35), a distance calculation (or sufficiently accurate one) may not be obtainable where the noise from the excitation pulse “merges” with relevant return signals. In some embodiments, the system 36 is programmed to determine when a transducer 18 falls within and/or approaches such a proximity threshold. While FIG. 2C only depicts one of 15A or 15B, each position, 15A or 15B, may have its own respective set of relative positions points (p1-p6) and radial distance measurements (D1-D6).

In response to determining that a transducer 18 falls within a proximity threshold, the system 36 causes the the one or more transducers 18 to excite at a lower frequency (e.g., half harmonic) that produces lower levels of excitation pulse noise. Based on return signals received using the lower frequency, the system 36 may calculate a distance measurement from the one or more transducers 18 to the structure wall 35 and use that calculation to determine the shape and/or dimensions of the structure such as further described herein.

In some embodiments, identifying structural features includes using another correlation model (e.g., based on a machine learning system such as a neural-network, K-nearest neighbor, Kernel estimation, Bayes classifier, Quadratic discriminant analysis, support vector machine, etc.) that characterizes one or more common shapes across each of the multiple cross-sectional shapes.

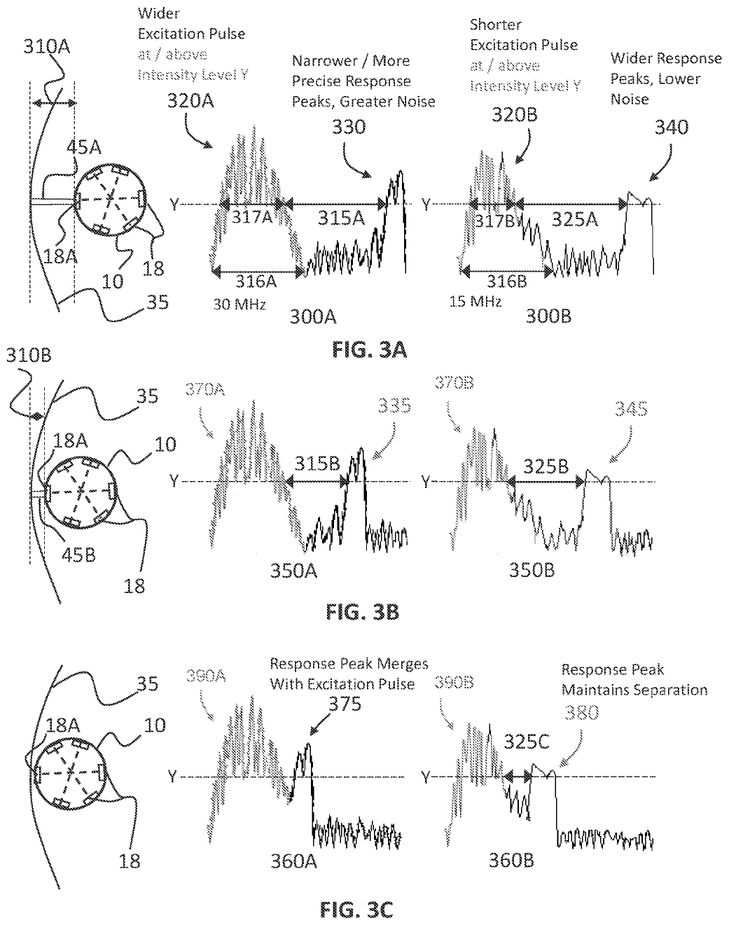

FIG. 3A shows a graphical illustration of ultrasound transducer signal intensities (Y) over time at different operating frequencies according to some embodiments. An ultrasound probe 10 with transducers 18 is shown positioned at a distance 310A from a structure 35. A transducer 18A directs signals 45A to structure 35 and receives return signals reflected from structure 35. An illustrative chart 300A of transducer 18A signal intensities over time activated at its fundamental resonant frequency (e.g., 30 MHz) shows distinct pulses/peaks, including the activation/excitation pulse 320A and reflected wall signal peak 330 separated by a time difference 315A. The time difference 315A may be used to calculate (orthogonal) distance 310A and determine a relative point of the structure 35 and, together with distances and points similarly calculated using other transducers 18, determine a cross-sectional shape representing structure 35 such as further described herein.

A chart 300B illustrates signal intensities from transducer 18A activated at a lower frequency (e.g., 15 MHz, half harmonic) than the fundamental harmonic frequency illustrated in chart 300A. Pulse 320B with width 316B (same width as 316A) shows a lower density of excitation/noise peaks than pulse 320A. In some embodiments, pulse width 316B can also have a shorter duration than the width 316A of pulse 320A. In certain embodiments, lowering the excitation frequency may lower the number of pulse cycles activating transducer 18A, even for an equal duration of time, thus bringing excitation signal intensity down, and subsequently making the excitation pulse total width shorter at/above an intensity level Y (e.g., at which other signals may be significantly interfered with). At intensity level Y, excitation pulse width 317B is shorter than pulse width 317A, therefore time interval 325A is wider than 315A for structure 35 located at a distance 310A. Furthermore, by lowering the excitation frequency, a main lobe of the ultrasound signal beam 45A gets wider while lowering the intensity of its side lobes, resulting in a wider (i.e., less precise), less noisy, and less intense wall signal peak 340 compared to wall signal peak 330 obtained at a higher excitation frequency. In some embodiments, the duration of ultrasound signal transmission at the second frequency is equal to the duration of ultrasound signal transmission at the first resonant frequency. In some embodiments, a particular intensity level, such as intensity level Y, is at least above a noise floor. In some embodiments, the particular intensity level is about −20 dB. In some embodiments, the particular intensity level is about −60 dB. In some embodiments, the particular intensity level is about −10 dB.

FIG. 3B shows a graphical illustration of ultrasound transducer signal intensities (Y) over time at different operating frequencies when the transducer is positioned closer to a structure relative to that shown in FIG. 3A. The ultrasound probe 10 with transducers 18 is shown positioned at a distance 310B from the structure 35, significantly closer to structure 35 compared to its position at distance 310A. Transducer 18A directs signals 45B to structure 35 and receives return signals reflected from structure 35. An illustrative chart 350A of signal intensities from transducer 18A activated at its fundamental frequency reflects less distinct and closer pulses/peaks compared to the peaks of FIG. 3A, as represented by time interval 315B.

A chart 350B illustrates signal intensities from transducer 18A activated at a lower frequency than the signal in chart 350A while the transducer 18A is positioned at distance 310B from structure 35. Chart 350B illustrates how a signal/structure peak 345 generated using a lower frequency may be wider and less precise than the signal/structure peak 335 of chart 350A. Chart 350B also illustrates how a excitation pulse 370B generated at a lower frequency may be narrower at or above a particular intensity level Y than the excitation pulse 370A of chart 350A generated at a higher frequency. The charts illustrate that the pulses/peaks (i.e. 370A, 335 and 370B, 345) are more distinguishable at a lower intensity as a time interval 325B is wider at the lower intensity in comparison to a time interval 315B at the higher intensity. In some embodiments, when the transducer 18A approaches or crosses within a particular distance threshold, the transducer 18A is transitioned to the lower frequency in order to better distinguish a structure peak. As described further herein, this may be accomplished by detecting a structure peak within or approaching a particular time period of the excitation pulse when operating at its fundamental frequency. In some embodiments, the transducer 18A is automatically transitioned to the lower frequency for every measurement and the resulting signal data is used to calculate a wall distance when it is determined that the higher frequency data provides inadequate coherence and differentiation between peaks.

In some embodiments, the variability, noise level, and/or other characteristics in the signal are used to identify a transition from a blood medium to a solid structure (e.g., vessel wall 35). For example, a lower noise level or variability (and increased stability) in the signal located after peaks (e.g., peaks 335 and/or 345) may be used to confirm that the peaks are associated with a structure wall signal peak. In some embodiments, the one or more transducers 18 generate lower noise levels in response to transmitting at the second resonant frequency compared to noise levels generated in response to transmitting at the first resonant frequency.

FIG. 3C is a graphical illustration of ultrasound transducer signal intensities (Y) over time at different operating frequencies when the transducer is positioned substantially adjacent to the structure shown in FIGS. 3A and 3B according to some embodiments. The ultrasound probe 10 with transducer 18A is shown adjacent structure 35, significantly closer to structure 35 compared to its position at distances 310A and 310B. An illustrative chart 360A of signal intensities from transducer 18A operating at its fundamental frequency reflects significantly indistinct or “merged” pulses/peaks 390A, 375 compared to the peaks and pulses of FIGS. 3A and 3B. Based on identifying a merger of the excitation pulse 390A and return signal structure peak 375 and/or an approach of the transducer to a structure (e.g., as illustrated in chart 350A), transducer 18A is transitioned to a lower operating frequency (e.g., half harmonic) and used to obtain additional signals.

A chart 360B illustrates signal intensities from transducer 18A activated at the lower frequency while the transducer 18A is positioned adjacent structure 35. Chart 360B illustrates how a structure peak 380 generated using a lower frequency may be distinguishable from an excitation pulse 390B at a time interval 325C even if the pulse 390A and return signal 375 are merged when the transducer operates at its fundamental frequency (e.g., as illustrated in chart 360A). That way, a measurement of the distance (even if very small) of the transducer 18A from the structure 35 can be measured. In some embodiments, when both fundamental and lower operating frequencies reflect a substantially merged pulse and return signal, it is determined that the distance between the one or more transducers 18 and structure 35 is at or around zero for purposes of calculating a point of the structure boundary (i.e., the transducer is directly adjacent the structure).

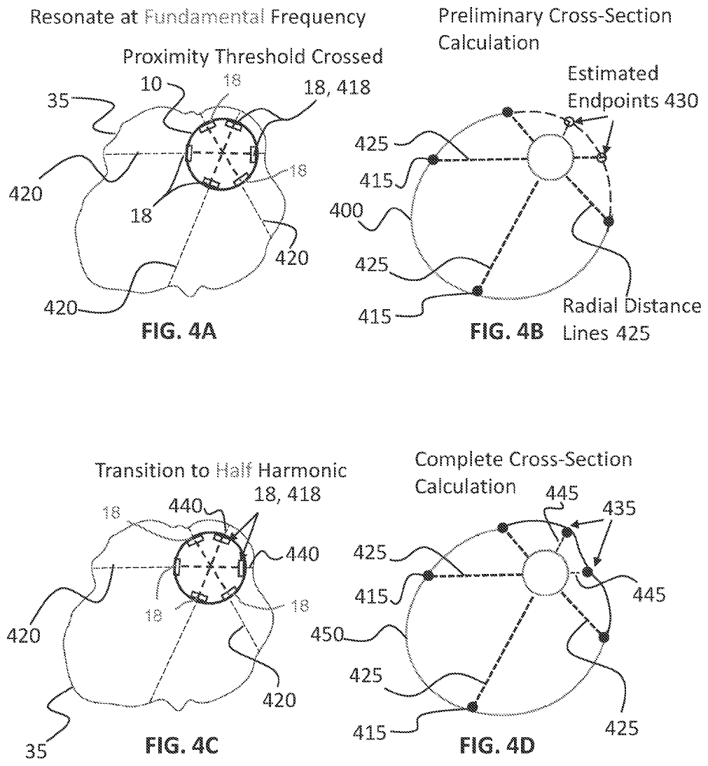

FIG. 4A is an illustrative cross-sectional diagram of an ultrasound transducer probe array positioned within a lumen structure while operating at fundamental frequency according to some embodiments. A probe 10 with an array of transducers 18 is positioned in a lumen structure 35. Two of the transducers 418 (i.e. a subset) are positioned where an orthogonal radial distance to the structure 35 is small enough to cause interference between an excitation pulse and a return signal pulse when the transducers 418 operate at their fundamental resonant frequency, thus significantly interfering with obtaining measurements based on those signals. The distances 420 from the other transducers 18 (i.e. another subset) of the probe's 10 array are sufficiently large so that such interference will not significantly impact distance measurements when the transducers 18 operate at their fundamental frequency.

FIG. 4B is an illustrative partially-calculated cross-sectional map of the lumen structure generated from distance measurements obtained through the ultrasound transducer probe of FIG. 4A according to some embodiments. A determination is made (e.g., through processing in system 36) of which transducers are not significantly interfered with by operating at their fundamental frequency. For example, significant noise interference may be associated with transducer proximity to the structure 35 such as further described herein.

In this example, it is determined that transducers 418 cannot obtain sufficiently distinguished signals for calculating distance measurements at their fundamental operating frequency while the remaining transducers 18 can. Signals from remaining transducers 18 are used to calculate radial distance lines 425 and endpoints 415. Based on these endpoints 415, a cross-section 400 of structure 35 is partially calculated that omits distance calculations from transducers 418.

Based on the partially-calculated cross-section 400, endpoints 430 of radial distance lines from transducers 418 to structure 35 may be estimated. For example, a curve-fit (e.g., splines) between endpoints 415 is used to partially calculate a cross-sectional shape of the structure 35. Then, radial distance lines between the transducers 418 and the partially calculated cross-sectional shape 400 of structure 35 are estimated. The lengths of these distance lines to endpoints 430 are used to determine whether transducers 418 are within a proximity threshold of structure 35. If it is determined that transducers 418 are within the proximity threshold, the transducers are transitioned to operating at a lower frequency (e.g., a half harmonic) in order to make additional distance calculations.

FIG. 4C is an illustrative cross-sectional diagram of an ultrasound transducer probe array positioned within a lumen structure while operating at a lower harmonic frequency relative to FIG. 4A according to some embodiments. FIG. 4D is an illustrative cross-sectional map image of the lumen structure generated from distance measurements obtained through the ultrasound transducer probe of FIG. 4C according to some embodiments. In response to the proximity threshold determination described with respect to FIGS. 4A and 4B, at least transducers 418 may be transitioned to operating at a lower frequency. After activating transducers 418 at the lower frequency, additional transducer signals are received and used to make distance calculations of radial distances 440 between respective transducers and structure 35. Radial distance lines 445 with endpoints 435 are calculated based on the distance calculations.

Based on a complete set of distance calculations and radial distance lines (e.g., utilizing all transducers 18 including transducers 418), a complete cross-sectional map image 450 is generated. Using the additional information from the distance lines calculated using the lower frequency, a more accurate representation of lumen 35 may be generated (e.g., in comparison to the partially calculated representation of FIG. 4B). While the transducers are divided into subsets (the two transducers 418 and the remaining six transducers 18) it is noted that any of the transducers may fall within one subset or another depending on the position of the probe 10 within the lumen 35. Further, each subset may include any number of transducers or proportion of transducers on the probe.

FIG. 5 is a flow chart of a process for utilizing multiple resonant frequencies for calculating structure dimensions according to some embodiments. At block 510, each of a plurality of transducers (e.g., of probe 10) are activated/excited at their fundamental (i.e., first harmonic) resonant frequency, by which they transmit ultrasound pulses, after which they receive responsive echo signals from surrounding media (e.g., blood) and structures/tissue (e.g., vessel wall). Responsive signals received by the transducers are converted into electrical signals that are obtained by a computer system (e.g., computer system 36).

At block 520, each of the plurality of transducers are activated/excited at a lower resonant frequency (e.g., half harmonic). At block 530, each of the sets of responsive signals are analyzed over intensity and time in order to make a determination of the distance between each transducer across a substantially perpendicular path to the structure. In some embodiments, the analysis identifies readily discernable peaks using the transducer's fundamental and/or lower operating frequency, reflecting that the structure is at a particular distance from the transducer based on time-of-flight calculations.

In some instances where both operating frequencies produce readily discernable peaks, and because the fundamental frequency may provide greater accuracy for distance calculations, the fundamental frequency signals are utilized without the lower frequency data to calculate distance. In some embodiments, where the distance is determined to exceed a particular value (e.g., a structure peak separated from an excitation peak by at least a certain predetermined value (e.g., in time)), it is determined that the fundamental frequency will be utilized to make the distance calculation and the lower frequency data omitted from the calculation.

In some embodiments, an analysis of the signals associated with the fundamental frequency reflect no discernable peak associated with the structure, indicating that the structure may be exceeding a particular distance from the transducer where structure-associated peaks are not readily discernable at the fundamental frequency (e.g., by being absorbed/attenuated by thick layers of intervening media (e.g., blood); since ultrasound signal attenuation in intervening media proportionally increases with frequency). Such analysis may direct the process in the use of the lower frequency data without the fundamental frequency data for calculating distance.

In some cases, it may not be possible to sufficiently distinguish the excitation pulses and structure signal peaks using either of the frequencies in order to make a distance calculation using time-of-flight analysis (e.g., where the excitation pulse and structure signal peak have “merged” as illustrated in FIG. 3C). In some embodiments, an absence of separate, distinct signal peaks associated with the structure indicate that the transducer (e.g., one or more transducers 18) is within a predetermined proximity of the structure (e.g., directly in contact). In some embodiments, a wider than typical excitation pulse is used to indicate that the structure is within the predetermined proximity. In such cases, the transducer may be treated as being directly adjacent to the structure (i.e., a distance of zero) along a perpendicular path, and a coordinate point is calculated accordingly at block 540. In some embodiments, the combinations of signals associated with both frequencies are determined to be inadequate for making calculations or reasonable assumptions pertaining to distances between the structure and respective transducers (e.g., from excessive noise).

At block 540, radial distances (i.e. 425) to the structure along respective perpendicular paths are calculated based on the analysis performed at block 530. Based on the distance calculations, coordinate points of the structure are calculated. In some embodiments, where the analysis at block 530 indicates that a reasonably accurate distance calculation or assumption is not obtainable with respect to certain transducers, the distance/location of the structure is estimated by interpolating between the calculated locations using signal data from the other transducers. These calculations may be used to determine a cross-sectional shape and/or various metrics of the structure (e.g., diameters, area, volume) such as further described herein. In some embodiments, the signals (i.e. pulses and peaks) that are determined to be more stable are used to calculate the respective distance.

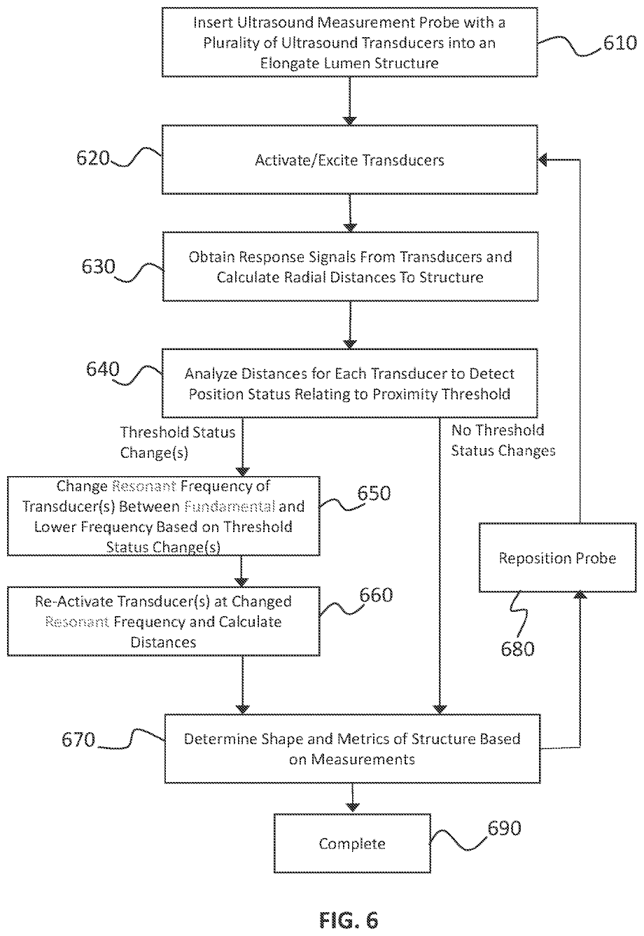

FIG. 6 is a flow chart of a process for transitioning transducers between high (i.e., fundamental/first harmonic) and lower resonant frequencies in response to monitoring their proximity to structures according to some embodiments. At block 610, an ultrasound probe (e.g., probe 10 of FIG. 1) is inserted into a structure (e.g., a blood vessel or other lumen), the probe having a plurality of ultrasound transducers (e.g. 18) circumferentially disposed about the probe. At block 620, after the probe is initially positioned to begin one or more in a series (i.e., a run) of cross-sectional measurements, the transducers are initially activated/excited at their high (i.e., fundamental/first harmonic resonant) operating frequency. At block 630, in response to signals transmitted from the transducers, response signals are reflected and received from surrounding media and structure (e.g., blood and blood vessel walls). Based on analyzing the signals, radial distance calculations between the transducers and structure are performed.

The distance calculations may be made by observing an excitation pulse and structure peak within the transducer signals. For example, the time interval between the excitation pulse and structure peak (i.e. of FIGS. 3A-3C) together with the speed of sound in the medium (e.g., blood) can be used to calculate their separation distance. In some instances, such as on account of noise and transducer proximity to structure, it is not possible to obtain a reasonably accurate distance measurement.

At block 640, distance calculations and/or other signal analysis is used to determine whether each of the transducers falls within or outside of a proximity threshold (and designated within first and second subsets of transducers, respectively). In some embodiments, such as where a direct distance calculation is not possible, the transducer signals are analyzed to detect a “merger” between the excitation pulse and a structure signal peak to indicate that the transducer is within the threshold such as described further herein. The status of whether each particular transducer falls within or outside of the proximity threshold is tracked (e.g., stored within computer memory). In some embodiments, the proximity threshold is about 0.3 millimeters. In some embodiments, the proximity threshold is about 0.2 millimeters. In some embodiments, the proximity threshold is about 0.1 millimeters.

In some embodiments, when at least one transducer is classified as being within the threshold, all of the transducers are excited/reactivated using a lower resonant frequency at block 660. Additional distance calculations may be made for those transducers where the fundamental resonant frequency permitted distance calculations. For example, the additional calculations at the lower frequencies may be used to verify the original calculations and/or used to obtain a set of points closer in time to each other (during which interval the probe will have been less likely to move).

In some embodiments, where it is not possible to directly calculate a radial distance for one or more transducers, a preliminarily calculated shape of the structure is used to determine an approximate position and distance(s) of the transducer(s). The preliminary shape calculation may be based on signals from transducers where a direct distance calculation is obtained. Based on the preliminary shape calculation (e.g., as described with respect to FIG. 4B), the distances of each of the transducers is calculated/estimated (including those where a direct distance could not be calculated).

In some embodiments, a machine learning system is programmed to identify/classify when and where transducer(s) are positioned within or outside of the proximity threshold of the structure. The system can be programmed with a machine learning model using signal data and/or independently calculated radial distances for transducers positioned proximate to a structure independently known/verified to be positioned either within or outside of a proximity threshold and, in some embodiments, more precisely where they are positioned. The machine learning system may utilize a neural-network, K-nearest neighbor, Kernel estimation, Bayes classifier, Quadratic discriminant analysis, support vector machine, and/or other methods.

In some embodiments, successive distance measurements for particular transducers are monitored to identify if the respective transducer is approaching (i.e., moving toward) a proximity threshold of the structure. By analyzing successive measurements and detecting that a subsequent measurement cannot be made for lack of a distinct structure peak, the transducer may be classified as having crossed the threshold until a subsequent measurement determines otherwise. In some embodiments, that transducer is operated at the lower frequency until it is determined that the transducer is no longer within the proximity threshold. When it is determined that the transducer is no longer within the threshold, the transducer is switched back to operating at its fundamental resonant frequency.

At block 650, in response to detecting changes in the status of transducer threshold proximity (e.g., between being inside/outside of the threshold to outside/inside), their operating frequency is changed accordingly. At block 660, transducers in which the status has changed are re-activated at the newly assigned operating frequency. For example, if the status of a transducer changes from being inside the threshold to being positioned outside of the threshold, the transducer is re-activated at the fundamental frequency at block 660 and vice versa. Based on signals obtained in response to being re-activated, additional calculations are made to determine radial distances between the transducer(s) and structure.

At block 670, utilizing response signals received from transducers using fundamental and/or lower frequencies, the shape and metrics of the structure are calculated (e.g., by system 36). If a run or series of cross-sectional measurements continues, the probe is re-positioned within the structure (e.g., using a pullback mechanism) at block 680 and the transducers are re-activated for further measurements at block 620. In some embodiments, based on their proximity threshold status determined at block 640, the transducers are activated at their fundamental or a lower frequency at block 620.

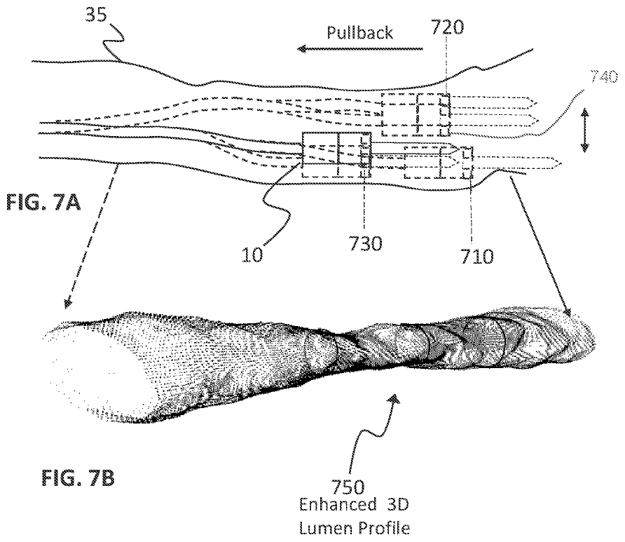

At block 690, the run or series of cross-sectional measurements is complete. The shape calculation(s) may be used to display a cross-sectional image map of the structure (e.g., as illustrated in FIG. 4D). Calculated metrics may include diameters (e.g., minimum and maximum diameters) and cross-sectional area of the structure. In some embodiments, multiple such cross-sectional calculations obtained at multiple longitudinal positions within the structure may be used to calculate and generate a three-dimensional shape/map (e.g., shown in FIG. 7B) of the structure and related metrics (e.g., volume).

FIG. 7A is an illustrative diagram of an ultrasound catheter probe 10 repositioned at multiple longitudinal positions 710, 720, 730 and 740 of a lumen 35 and multiple positions within each cross section of the multiple longitudinal positions. In some embodiments, the probe 10 is moved longitudinally within the lumen 35 (e.g., by way of a “pullback” operation). At the different positions 710, 720, 730 and 740, a set of distance measurements using the probe 10 is obtained (e.g., as described further herein). The different positions shown can additionally include different lateral positions within the same cross-section of a lumen (e.g., as shown in FIGS. 2A-2B and 3A-3C) and/or rotational positions. These different positions may be influenced by manual/robotic actuation and/or movement caused by the structure (e.g., blood flow/heart beating). The dynamic adjustment of the transducer operating frequency described herein permits such movement/positioning of the probe and ability to obtain more complete and accurate cross-sectional calculations when the probe transducers' outward radial proximity is in contact or close contact with the structure.

FIG. 7B is an illustrative diagram of a three-dimensional mapping 750 of a lumen based on ultrasound measurements obtained at the multiple catheter probe positions of FIG. 7A. Based on sets of distance measurements obtained by an array of circumferentially arranged transducers, cross-sectional shapes of the lumen 35 may be calculated such as further described herein. The cross-sectional shapes calculated for different longitudinal positions may be combined to calculate three-dimensional shapes and dimensions (e.g., volumes) of the structure.

As described herein, generating a map image of the structure on a computer may include calculating and plotting a cross-section of the structure or a three dimensional model of the structure based on the above described processes.

The processes described herein (e.g., the processes of FIGS. 5 and 6) are not limited to use with the hardware shown and described herein. They may find applicability in any computing or processing environment and with any type of machine or set of machines that is capable of running a computer program. The processes described herein may be implemented in hardware, software, or a combination of the two. The processes described herein may be implemented in computer programs executed on programmable computers/machines that each includes a processor, a non-transitory machine-readable medium or other article of manufacture that is readable by the processor (including volatile and non-volatile memory and/or storage elements), at least one input device, and one or more output devices. Program code may be applied to data entered using an input device to perform any of the processes described herein and to generate output information.

The processing blocks (e.g., in the processes of FIGS. 5 and 6) associated with implementing the system may be performed by one or more programmable processors executing one or more computer programs to perform the functions of the system. All or part of the system may be implemented as special purpose logic circuitry (e.g., an FPGA (field-programmable gate array) and/or an ASIC (application-specific integrated circuit)). All or part of the system may be implemented using electronic hardware circuitry that include electronic devices such as, for example, at least one of a processor, a memory, a programmable logic device, and/or a logic gate.

The processes described herein are not limited to the specific examples described. For example, the processes of FIGS. 5 and 6 are not limited to the specific processing orders illustrated. Rather, any of the processing blocks of FIGS. 5 and 6 may be re-ordered, combined or removed, performed in parallel or in serial, as necessary, to achieve the results set forth above.

Elements of different embodiments described herein may be combined to form other embodiments not specifically set forth above. Various elements, which are described in the context of a single embodiment, may also be provided separately or in any suitable sub-combination. Other embodiments not specifically described herein are also within the scope of the following claims.

1. A method of ultrasound measuring, the method comprising:
transmitting ultrasound signals toward a structure from each of a plurality of ultrasound transducers centered at a first resonant frequency and centered at a second resonant frequency, the second resonant frequency lower than the first resonant frequency;
receiving ultrasound signals from the structure responsive to the transmitted ultrasound signals;
determining distances between each of the plurality of transducers and the structure based on the received ultrasound signals;
for each determined distance, calculating a circumferentially separated coordinate point of the structure based on the respective determined distance between the respective transducer and structure; and
generating a computed map image in a computer display that comprises calculating and plotting a cross-sectional map of the structure by interpolating between the circumferentially separated coordinate points of the structure each based on the respective determined distance.

2. The method of claim 1 wherein the first resonant frequency is a fundamental/first harmonic of the transducers.

3. The method of claim 1 wherein the second resonant frequency is a half harmonic of the transducers.

4. The method of claim 1 wherein the first resonant frequency is about 30 MHz and the second resonant frequency is about 15 MHz.

5. The method of claim 1 further comprising:
determining that one or more of the plurality of transducers is within a proximity threshold of the structure; and
in response to determining that the one or more of the plurality of transducers are within the proximity threshold, determining the distances between the one or more transducers and the structure based on the ultrasound signals responsive to the second resonant frequency.

6. The method of claim 5 wherein determining that one or more of the plurality of transducers is within the proximity threshold comprises identifying an absence of a structure signal peak separated by more than a predetermined time interval from an excitation pulse signal.

7. The method of claim 5 wherein determining that one or more of the plurality of transducers is within the proximity threshold comprises:
based on the received ultrasound signals, calculating a distance between each of a first subset of the plurality of transducers and the structure;
for each calculated distance, calculating the circumferentially separated coordinate point of the structure based on the respective determined distance between the respective transducer and the structure;
determining a partially-calculated cross-section of the structure based on the separated coordinate points for the first subset of transducers;
estimating a distance between each of a second subset of the plurality of transducers and the structure based on the partially-calculated cross-section, the second subset comprising one or more transducers not within the first subset; and
determining that the second subset of transducers is within the proximity threshold based on the estimation of their distance from the structure.

8. The method of claim 7 wherein estimating the distance between each of the second subset of transducers and the structure comprises calculating a length of a radial distance line between a position of each of the subset of transducers and the structure based on the partially-calculated cross-section.

9. The method of claim 5 wherein the proximity threshold is about 0.3 millimeters or less.

10. The method of claim 5 further comprising:
determining that one or more of the plurality of transducers is not within the proximity threshold of the structure; and
in response to determining that one or more of the plurality of transducers is not within the proximity threshold of the structure, calculating the distances between the transducers not within the proximity threshold and the structure based on the first resonant frequency.

11. The method of claim 1 wherein calculating a distance between each of the plurality of transducers and the structure is based on the ultrasound signals received from the structure in response to a combination of the ultrasound signals transmitted at the first resonant frequency and the second resonant frequency.

12. The method of claim 11 wherein a distance between a transducer and the structure is calculated to be about zero based on determining, from the received signals, that the transducer is within a proximity threshold of the structure.

13. The method of claim 12 wherein determining that the transducer is within the proximity threshold comprises determining that a wall signal peak within the received signals has substantially merged with an excitation pulse of the received signals.

14. The method of claim 11 wherein calculating a distance between a transducer and the structure is based on determining the stability of the signals using each of the first and second resonant frequencies received from the structure and selecting the signals determined to be more stable to calculate the respective distance.

15. The method of claim 11 wherein calculating a distance between a transducer and the structure is based on signals of at least one of the first or second resonant frequencies and by verifying the distance calculation using signals of the other of the at least one of the first or second resonant frequencies.

16. The method of claim 11 wherein calculating a distance between each of the transducers and the structure is based on:
in response to determining that the respective transducer is within a proximity threshold of the structure, calculating the distance based on signals using the second resonant frequency; and
in response to determining that the respective transducer is not within the proximity threshold of the structure, calculating the distance based on signals using the first resonant frequency.

17. The method of claim 1 wherein a duration of ultrasound signal transmission at the second resonant frequency is equal to a duration of ultrasound signal transmission at the first resonant frequency.

18. The method of claim 1 wherein a duration of ultrasound signal transmission at the second resonant frequency is no greater than about a duration of ultrasound signal transmission at the first resonant frequency.

19. The method of claim 1 wherein one or more transducers generate lower noise levels in response to transmitting at the second resonant frequency compared to noise levels generated in response to transmitting at the first resonant frequency.

20. The method of claim 1 wherein transmitting the ultrasound signals comprises generating a main ultrasound beam and side lobes of the main beam, wherein the main beam is wider and the side lobes less intense using the second resonant frequency compared to using the first resonant frequency.

21. The method of claim 1 wherein transmitting the ultrasound signals at the second resonant frequency results in an excitation pulse width that is narrower at and above a particular intensity level compared to transmitting the ultrasound signals at the first resonant frequency.

22. The method of claim 21 wherein the particular intensity level is at least above a noise floor.

23. The method of claim 21 wherein the particular intensity level is about −100 dB or greater.

24. The method of claim 21 wherein generating a narrower excitation pulse at and above the particular intensity level comprises transmitting the ultrasound signals at a lower intensity for the second resonant frequency compared to the first resonant frequency.

25. The method of claim 21 wherein generating a narrower excitation pulse at and above the particular intensity level comprises transmitting the ultrasound signals with a lower number of excitation signal pulses for the second resonant frequency compared to the first resonant frequency.

26. The method of claim 1 wherein:
each of the plurality of ultrasound transducers is circumferentially separated from each other;
transmitting ultrasound signals toward the structure from each of the plurality of ultrasound transducers comprises transmitting substantially orthogonally a signal from each transducer toward a respectively separated circumferential portion of the structure substantially parallel to the transducer at the first resonant frequency and at the second resonant frequency; and
receiving ultrasound signals from the structure responsive to the transmitted ultrasound signals comprises receiving at each separated transducer a reflected signal from the respectively circumferentially separated section of the structure.

27. An ultrasound system for measuring dimensions of a structure, the system comprising:
a flexible body elongated along a longitudinal axis and assembled for insertion into the structure;
a plurality of ultrasound transducers arranged on the flexible body; and one or more processors programmed and configured to cause:
transmitting ultrasound signals from the plurality of ultrasound transducers of an ultrasound probe toward the structure at a first resonant frequency; and
receiving responsive ultrasound signals at the ultrasound transducers responsive to respective sets of the transmitted ultrasound signals; and
in response to determining that one or more of the plurality of transducers is within a proximity threshold:
transmitting ultrasound signals from the one or more transducers toward the structure at a second resonant frequency lower than the first resonant frequency;
receiving ultrasound signals from the structure responsive to the transmitted ultrasound signals at the second resonant frequency; and
calculating a distance between each of the plurality of transducers and the structure based on the ultrasound signals received from the structure in response to the transmitted ultrasound signals at the first resonant frequency and/or second resonant frequency.

28. An ultrasound system for measuring the dimensions of a structure, the system comprising:
a flexible body elongated along a longitudinal axis and assembled for insertion into the structure;
a plurality of ultrasound transducers arranged on the flexible body; and
one or more processors programmed and configured to cause:
transmitting ultrasound signals toward a structure from each of a plurality of ultrasound transducers centered at a first resonant frequency and centered at a second resonant frequency, the second resonant frequency lower than the first resonant frequency;
receiving ultrasound signals from the structure responsive to the transmitted ultrasound signals;
determining distances between each of the plurality of transducers and the structure based on the received ultrasound signals;
for each determined distance, calculating a circumferentially separated coordinate point of the structure based on the respective determined distance between the respective transducer and structure;
generating a computed map image in a computer display that comprises calculating and plotting a cross-sectional map of the structure by interpolating between the circumferentially separated coordinate points of the structure each based on the respective determined distance; and
generating the computed map image in the computer display that comprises combining and plotting a series of multiple previously calculated cross-sectional maps at multiple longitudinal and lateral positions within the structure to generate a three-dimensional mapping representation of the structure.
